(19) United States
(12) Patent Application Publication
(10) Pub. No.: US 2010/0298947 A1
(43) Pub. Date: Nov. 25, 2010

(54) Precision Total Knee Arthroplasty

(76) Inventor: Anthony S. Unger, Bethesda, MD (US)

Correspondence Address: Perry E. Van Over;PERRY E. VAN OVER & ASSOCIATES, P.O. Box 399, Fairfax, VA, 22038-0399, US

(21) Appl. No.: 12/428,448

(22) Filed: Apr. 22, 2009

(51) Int. Cl. A61F 2/38; B29C 43/18

(52) U.S. Cl. 623/20.32; 264/269

(57) ABSTRACT

Provided is a novel modular tibial prosthesis that includes an articulation component formed by direct pressure molding of a resin composition into a tibial tray component to provide a factory formed resin-metal composite structure a tibial tray module. The tibial tray component being capable of releasable attachment to an underlying keel element. A method of manufacture of the tibial prosthesis is also disclosed.

### BACKGROUND OF THE INVENTION

1. Technical Field

The present invention relates to orthopedic surgery and in particular to the provision and implantation of a modular tibial prosthesis for use in a total knee arthroplasty procedure. More particularly the present invention relates to a modular tibial prosthesis, the components of which can be selectively combined as necessary to meet the anatomical and therapeutic requirements of the patient.

2. Background Art

Total knee arthroplasty is a surgical procedure for replacing a patient's injured or damaged knee joint with an artificial knee joint. This joint replacement procedure can also be used to relieve pain in a knee joint damaged by osteoarthritis or rheumatoid arthritis. Total knee replacement can also be employed to ameliorate the discomfort that can result from deformed and unstable knees, cartilage destruction, and severe patellofemoral arthritis.

A typical total knee replacement procedure includes the implantation of a femoral prosthetic component, a patella prosthetic component, and a tibial prosthetic component. The femoral component will generally includes a pair of laterally spaced apart condylar portions, the distal surfaces of which articulate with complementary configured condylar articulating surfaces formed on the upper surface of the tibial prosthetic component. The tibial prosthetic component can be of a one-piece, mono-block design or it can be a more preferred modular design. Modular designed devices enable the surgeon to trial different components during the course of the procedure before final implanting of the tibial prosthesis. Further, if a revision of the implant is later found to be necessary the modular design facilitates replacement of worn or damaged components as compared to the more extensive procedures required for explanation of a mono-block design prosthesis.

The conventional modular tibial prosthetic device includes an upper portion, the tibial tray insert, which is typically configured to have an upper surface with two parallel concavities that are complimentary in shape to the condylar portions of the femoral prosthetic component. Tibal tray inserts are generally manufactured of polyethylene or similar synthetic resin materials and are sized and configured to be inserted into the upper surface area of a tibial tray or base plate portion of the tibial prosthetic device at the time of the surgery. The metal, metal-alloy, or composite material tibial tray portion is generally manufactured of a strong, biocompatible material such as surgical grade stainless steel, titanium, chrome-cobalt, and the like. More recently, proprietary materials, such as Tritanium™ (Stryker Orthopaedics, Mahwah, N.J.) and Trabecular Metal™ (Zimmer Holdings, Inc., Warsaw, Ind.) have been used with success in the manufacture of porous surfaced orthopedic implants. Porous materials of this sort can be employed to provide strong yet extremely porous-surfaced implants that facilitate the in-growth of surrounding tissue into the implant. The resin tibial tray insert provides a smooth, durable bearing surface for the articulating motion between the condylar surfaces of the femoral component and the upper surface of the underlying tibial tray, which is attached to the prepared upper surface of the patient's tibia. Typically the tibial tray portion of the tibial prosthetic component includes the use of use a stem or keel, which descends from the underside of the tibial tray and is embedded into a prepared cavity in the upper surface of the tibia. This stem can be integral with the tibial tray or it can be a separate piece that is attached by screws for example.

An on-going problem has been the failure to provide a secure fit of the polyethylene tibial tray insert onto the upper surface of the underlying tibial tray. As demonstrated in U.S. Pat. No. 4,938,769 issued to Shaw, conventional modular tibial prosthetic devices are designed such that the tibial tray portion of the device is first attached to the prepared upper surface of the tibia and the polyethylene insert is then manually inserted by the surgeon into position in the tibial tray. This manual insertion of the polyethylene insert into the tibial tray of conventional modular prosthetic devices by the surgeon has consistently resulted in a less-than-perfect tit between the two components that, over a period of time, allows for micro-motion of the softer insert against the metallic surface of the tibial tray. Repeated studies have shown that virtually all conventional tibial tray inserts undergo some degree of backside wear due to the micro motion of the tibial tray insert relative to the tibial tray. Backside wear in the form of abrasion, scratching, pitting, burnishing, delamination, protrusions, and polyethylene micro-debris can all be observed over time in conventional total knee replacements (The Journal of Bone and Joint Surgery (American) 86:305-311 (2004)). Additional studies have found that backside wear can contribute up to 30% of the total wear effects on the tibial tray insert (Journal of Bone and Joint Surgery—British Volume, Vol 87-B, Issue SUPP III, 337-338). The shortened useful life of the tibial tray insert due to the effects of backside wear is a problem for which a solution is badly needed; however, the deleterious effect of osteolysis caused by backside wear debris is a health risk to the patient and therefore represents a more serious problem in need of a remedy. The degree of tibial insert backside wear can be exacerbated by the use of tibial trays that are secured to the underlying tibia by screws inserted through screw holes in the bottom of the tibial tray. In procedures using cement to aid in securing the tibial tray insert to the tibial tray, an increase in micro debris has been observed.

There exists therefore a need to provide a modular tibial prosthetic device for use in total knee replacement that absolutely secures the tibial tray insert onto the upper surface of the tibial tray so as to eliminate micro motion between the polyethylene insert and the underlying tibial tray thus avoiding the deleterious effects of backside wear. Additionally, such a device should be provided with a modular design that permits the surgeon to combine the components of the tibial prosthesis to best fit the needs of the patient and to easily replace worn tibial tray inserts when such revision is required.

### SUMMARY OF THE DISCLOSURE

The present invention meets the above identified need by providing a novel modular tibial prosthesis for use in a total knee arthroplasty, wherein the tibial prosthesis includes a tibial tray module and a keel element, the tibial tray module being factory manufactured as a resin-metal composite modular unit having two components, a resin based articulation component and a metal or metal alloy tibial tray component.

Also provided is a novel modular tibial prosthesis having a tibial tray module wherein the articulation component is factory-formed by direct compression molding of a resin composition into the upper recess of the tibial tray component.

Also provided is a novel modular tibial prosthesis wherein the undersurface of the tibial tray component is porous and capable of promoting ingrowth of bone.

Also provided is a novel modular tibial prosthesis wherein the tibial tray component is secured to the keel element by at least one trunnion descending from the underside of the tibial tray component and configured to compression fit into at least one correspondingly sized and configured trunnion receptacle defined in the upper surface of the keel element.

Also provided is a novel modular tibial prosthesis wherein the tibial tray component includes an upwardly directed tray side wall that is circumferentially disposed and upwardly directed around the lateral limit of the tibial tray component upper surface, the tray side wall including an inwardly directed retaining lip sized and configured to securely seat within a circumferentially disposed retaining groove defined about the lateral surface of the articulation component, the retaining groove being formed by direct compression molding of the articulation component into the tibial tray component.

Also provided is a novel modular tibial prosthesis having a factory formed resin-metal composite module that includes a resin articulation component and a metal or metal alloy tibial tray component, the tibial tray module being attachable to a keel element wherein at least one osteotome receptacle is defined in the side of the module or the keel element at the juncture of the tibial tray module and the keel element, the osteotome receptacle being sized to permit a portion of the osteotome to be inserted into the receptacle to facilitate mechanical leverage separation of the module from the attached keel element.

Also provided is method of manufacturing a novel modular tibial prosthesis wherein the tibial prosthesis includes factory formed tibial tray module that is formed of an articulation component and a tibial tray component, and a keel element that is attachable to the underside of the tibial tray module.

Also provided is a method of manufacturing a tibial tray module for a novel modular tibial prosthesis wherein the articulation component is factory-formed directly on the upper surface of the tibial tray component by direct compression molding.

Also provided is a method of implanting a modular tibial prosthesis, the method including providing a tibial prosthesis having a factory formed composite tibial tray module that includes an articulation component and a tibial tray component with an attachable keel element, precision machining of the tibial bone in preparation to receive the modular tibial prosthesis, and implanting the modular tibial prosthesis.

Also provided is a kit containing a novel tibial prosthesis.

### BRIEF DESCRIPTION OF THE DRAWINGS

The foregoing and other features of the disclosed device will become apparent to one skilled in the art to which the present invention relates upon consideration of the following description of exemplary embodiments with reference to the accompanying drawings, wherein:

FIG. 1 shows an exploded view of the novel modular tibial prosthesis of the present invention in an assembly alignment for a total knee arthroplasty.

FIG. 2 shows a top view of the tibial tray component of the tibial tray module of the present invention.

FIG. 3 shows a bottom view of the tibial tray component of the tibial tray module of the present invention.

FIG. 4 shows a front view of the tibial tray component of the tibial tray module of the present invention.

FIG. 5 shows a perspective view of the tibial tray module having an articulation component factory formed by direct compression molding into the upper portion of the tibial tray component.

FIG. 6 shows a side view of the tibial tray module including an articulation component factory formed by direct compression molding into the upper portion of the tibial tray component.

### DETAILED DESCRIPTION OF PREFERRED EMBODIMENTS

Detailed embodiments of the present invention are disclosed herein; however, it is understood that the following description and each of the accompanying figures are provided as being exemplary of the invention, which may be embodied in various forms without departing from the scope of the claimed invention. Thus; the specific structural and functional details provided in the following description are non-limiting, but serve merely as a basis for the invention as defined by the claims provided herewith. The device described below can be modified as needed to conform to further development and improvement of materials without departing from the inventor's concept of the invention as claimed.

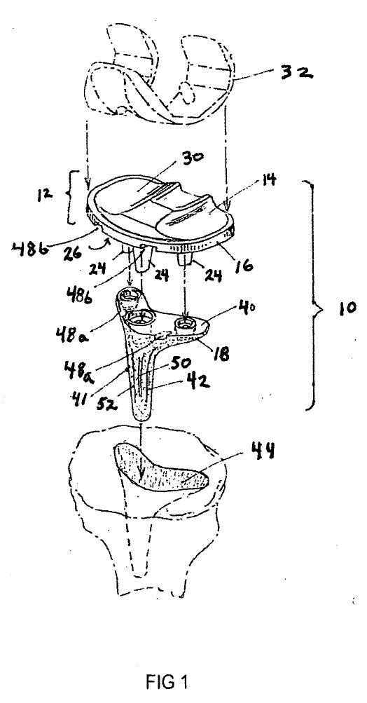

The novel modular tibial prosthesis device, as generally shown at 10 in FIG. 1, includes a tibial tray module 12, which is formed by direct pressure molding of an articulation component 14 into a tibial tray component 16. The tibial tray module 12, unlike conventional tibial prosthetic devices, is not an assembly of separately manufactured and finished parts. Rather, the tibial tray module 12 of the present invention is a factory manufactured resin-metal composite module in which an unformed and uncured resin composition is placed in a pre-formed tibial tray recess 20 on the upper side of a metallic tibial tray component 16 and by the process of direct pressure molding is formed into a unitary composite module having an upper articulation component 14 and a lower tibial tray component 16. Also provided is a metal or metal alloy keel element 18, which is configured to be releasably attached to the underside of the tibial tray module 12 in the surgical procedure of a total knee replacement. The keel element 18 is configured to be implanted in the upper portion of a subject's tibia and subsequently, securely attached to the tibial tray module during the surgical implantation procedure. However, if a revision of the tibial prosthesis device 10 is later required, the tibial tray module 12 can be selectively disengaged from the underlying keel element 18. To best promote a strong hold between the keel element 12 and the tibial bone of a subject, the keel element 18 is preferably configured to have a cruciate like shape with the apex being anterior as shown in FIG. 1.

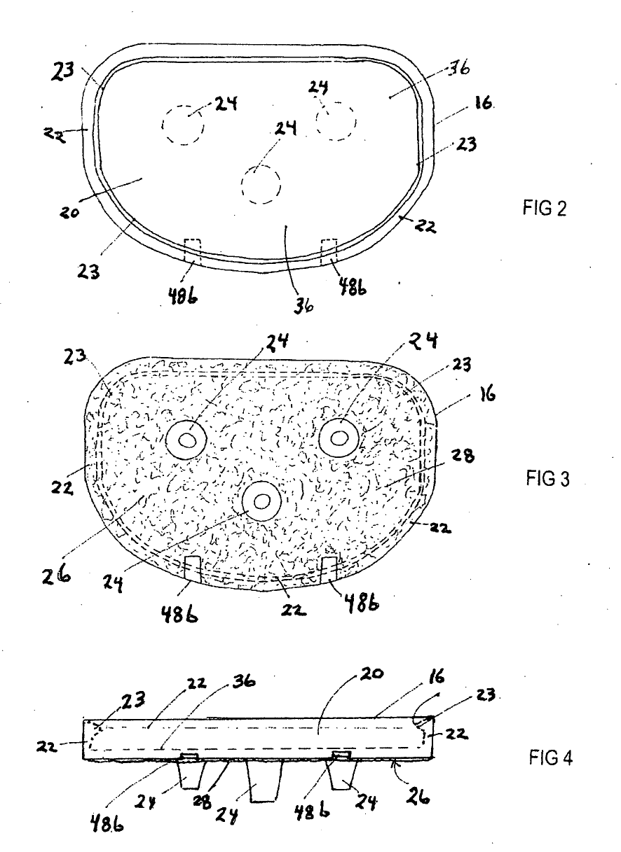

As shown in FIGS. 2, 3, and 4 the tibial tray component 16 includes a tibial tray recess 20 defined within the circumferentially disposed and upwardly directed tray wall 22. A retaining ridge 23 extends inwardly from the upper portion of the tray wall 22. This retaining ridge 23 is disposed circumferentially around at least a portion of the inner surface of the tray wall 22. Preferably, the retaining ridge 23 will be fully disposed around the inner circumference of the tray wall; however, it is within the concept of the invention that less that the full circumference of the tray wall 22 will define the inwardly directed retaining ridge 23.

Extending downwardly from the underside of the tibial tray component 16 are multiple, preferably three, trunnions 24, which provide a securing mechanism for the tibial tray module 12 when connected to the keel element 18 of the prosthesis device 10. The metal or metal alloy components of the tibial prosthesis device 10, the tibial tray component 16 and the keel element 18 can be made of any acceptable material known in the art, such as, for example, chrome-cobalt, stainless surgical steel, titanium and the like. The under surface 26 of the tibial tray component 14 is coated with a porous layer 28 that serves to promote bone growth into the pores of the material thus providing the primary means of securing the tibial tray component 16 to the upper prepared surface of the tibia. A preferred material for the porous layer 28 is tritanium (tantalum over carbon) or Trabecular Metal™ (Zimmer Holdings, Inc., Warsaw, Ind.).

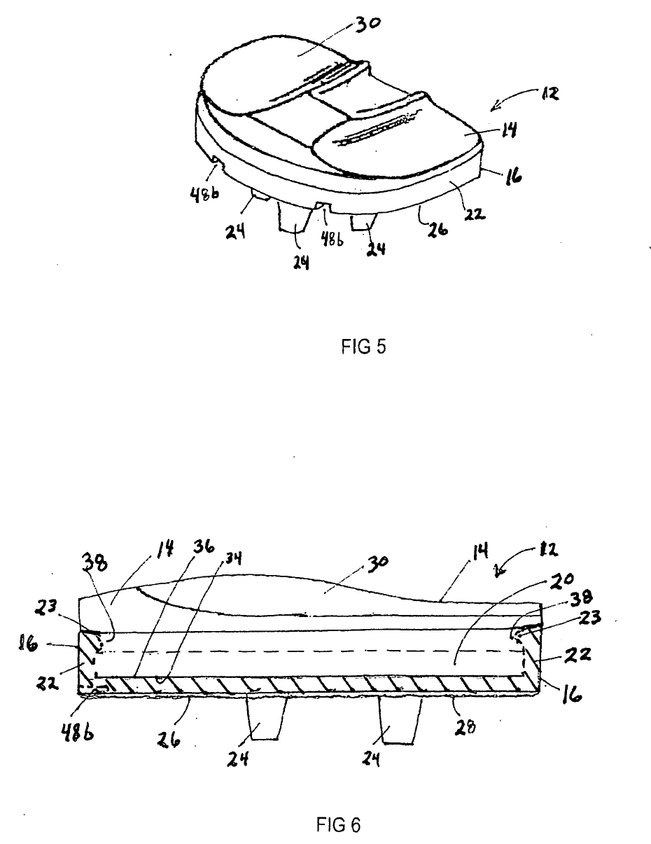

The articulation component 14, as shown in FIGS. 1, 5, and 6, which is formed by direct pressure molding into the tibial tray recess 20 of the tibial tray component 16, is configured to have a somewhat concave upper surface 30. This concave upper surface 30 is intended to simulate the natural shape of the condyles of the tibia and, as such, serves to cooperate with the condylar of the femoral component 32 so that the articulation of the knee after the total knee arthroplasty simulates the articulation of the natural human knee. As shown in FIGS. 5 and 6, the under surface 34 of the articulation component 14 is by the process of direct pressure molding within the tibial tray recess 20 formed into an exactly complementary shape to the upper surface 36 of the tibial tray recess 20. Conventional tibial prosthetic devices represent attempts to separately manufacture individual resin and metal components of tibial prosthetic devices so as to have an approximate complementary fit to each other. In contrast the present invention, being a direct pressure molded unitary resin-metal module, achieves a perfectly complementary fit of the components. Having a perfectly complementary fit of the resin-based articulation component 14 and the metallic tibial tray component 16, the tibial tray module 12 avoids the deleterious effects of backside wear that results from micro motion between the resin and metal components in conventional tibial prosthetic devices. The present invention's advantage of eliminating micro motion and backside wear cannot be duplicated by conventional devices, which by design and separate component manufacturing techniques are incapable of achieving a perfectly complementary fit.

As shown in FIGS. 5 and 6 the process of direct pressure molding of the articulation component 14 into the tibial tray recess 20 of the tibial tray component 16 creates a locking groove 38 defined around the outer edge of the articulation component 14 that is perfectly complementary in size and shape to the retaining ridge 22 of the tibial tray component 16. The articulation component 14 is preferably manufactured using a resin composition, such as polyethylene; however, any strong durable, biocompatible material capable of being formed by a direct pressure molding process and suitable for use as a joint articulation surface can be used without departing from the concept of the present invention.

Unlike conventional tibial tray prosthetic devices, which are assembled by manually inserting a pre-formed plastic articulating surface insert into a metallic tray, the tibial tray module 12 of the present invention is produced by providing an unformed, uncured resin composition into the tibial tray recess 20 of the tibia tray component 16 and molding by direct pressure a resin-metal unitary tibial tray module. The process of manufacturing resin-based parts by direct pressure molding is known and any variation of that process can be employed in the process of manufacturing the tibial tray module 12 of the present invention.

The keel element 18 is an essential element of the tibial prosthesis device 10; however, it is employed primarily as a means of initially securing the tibial prosthesis device 10 to the tibia and unlike conventional tibial prosthetic devices is not relied upon for the primary security of the tibial prosthesis to the upper surface of the tibia. It is the purpose of the keel element 18 to firmly secure the tibial prosthesis device 10 to the patient's tibia long enough for bone growth to occur between the upper surface of the tibia and the porous layer 28 coating the under surface of the tibial tray component 16. It is this bone growth into the porous surface 28 that provides the long term security of the tibial prosthesis device 10 to the tibia. The keel element 18, as shown in FIG. 1, is configured to have an upper flat surface 40 and an underlying downward taper portion 41. Similar to conventional keels or stems known in the art, the taper portion 41 of the keel element 18 of the present invention is sized and configured to closely fit within a calcar recess 44 prepared in the upper surface of the tibia. In the present invention, the keel element preferably is finished with a smooth surface as the long term fixation of the tibial prosthesis device 10 to the tibia is not dependent upon the attachment of the keel element 18; but, rather depends primarily on the ingrowth of bone into the porous layer 28 on the undersurface 26 of the tibial tray component. Although not preferred, it is possible that the downward tapering portion 41 of the keel element 18 can be coated with a highly porous material, such as, for example, Tritanium™ (cited above), Trabecular Metal™ (cited above), or the like. As shown in FIG. 1, alternatively the downward tapering portion 41 of the keel element 16 can also be fluted 50, that is, the surface of the keel element 16 can be provided with multiple longitudinal ridges and grooves to increase the total surface area in contact with new bone growth in the calcar recess 44 of the tibia.

Defined in the upper surface 40 of the keel element 18 are multiple, preferably three, trunnion receptacles 46 that correspond in size, shape, and relative position to complement the trunnions 24 which project downward from the under surface 26 of the tibial tray component 16. In the process of implanting the tibial prosthesis device 10 of the present invention, the Morse taper of the tibial tray component trunnions 24 when impacted into the corresponding trunnion receptacles 46 of the keel element form a cold weld, which securely holds the tibial tray component 16 to the keel element 18. As is known in the art, the formation of such a cold weld connection can be extremely secure. However, in the eventuality that a revision of the total knee arthroplasty is required, the present invention provides a device and method by which the tibial tray component 16 can, if required, be separated from the keel element 18. At least one keel separating notch 48a, as shown in FIG. 1, can be defined at a point along the edge of the upper surface of the keel element 18. Alternatively or in combination with the use of a keel separating notch 48a defined in the keel element, a keel separating notch 48b can be defined at a point along the edge of the under surface 26 of the tibial tray component 16. In either or both alternative configurations of the keel separating notch 48a or 48b, an osteotome, sized and configured to fit into the space provided by the keel separating notch 48a or 48b, can be employed to facilitate prying apart the cold welded tibial tray component 16 from the keel element 18. By this process, a revision of the tibial prosthesis device 10 can be performed while leaving the keel element 18 in place within the calcar recess of the tibia.

Similar to conventional methods of preparing the tibia to receive a tibial prosthesis, in the practice of the present invention the tibia of the patient can be prepared to receive the modular tibial prosthesis device 10 described and claimed herein. In this preparation process an initial or rough cut of the upper condylar surface of the tibia is accomplished using an external guide. Additional bone can be resected as necessary at that time. A trial tibial tray, as is known in the art, can then be employed in preparation for forming the calcar recess 44 in the upper surface of the tibia. U.S. Pat. No. 7,390,327 issued to Callazo et al. and U.S. Pat. No. 5,976,147 issued to LaSalle et al., the disclosures of which are fully incorporated herein by reference, describe the use of a punch apparatus useful for preparing a bone to receive an implant such as a keel element of the present invention. A trial keel can be inserted into the calcar recess 44 and used as a guide for a tibial machiner or miller as is well known in the art for preparing an absolutely smooth upper surface of the tibia. Having prepared the calcar recess 44 and the surface of the tibia, the trial keel is replaced with the keel element 18 of the present invention. An impactor, as is commonly used in the art, can be employed to drive the keel element 18 into position within the calcar recess 44 of the tibia. At this time, the tibial tray module 12 is aligned such that the downwardly directed multiple trunnions 24 of the tibial tray component 16 are positioned over the corresponding multiple trunnion receptacles 46 of the keel element 18. The tibial tray module 12 is then firmly connected to the keel element 18 by impacting the trunnions 24 into the trunnion receptacles 46 to form a cold weld connection. At this time it is possible the keel element 18 connected to the tibial tray module 12 can be further driven into the calcar recess 44 of the tibia. Although the present invention is preferably provided as a tibial prosthesis capable of being securely affixed to the tibia of the patient without the need for cement, it is possible that, as determined necessary by the surgeon, cement can be employed between the underside of the tibial tray component 16 of the tibial tray module 12 and the tibia. In the process of providing the present invention and implanting the same in a patient, the tibial tray module 12 can be offered as a cruciate-retaining or posterior-stabilized total knee replacement without departing from the concept of the invention.

The inventor has contemplated that each of the components of the present invention can be provided in different sizes and configured so as to be interchangeable thus permitting the surgeon to make a best fit for the particular anatomy of the patient being treated.

The device 10 can be manufactured as integral components by methods known in the art, to include, for example, molding, casting, forming, extruding, and machine processes. The components can be manufactured using materials having sufficient strength, resiliency and biocompatibility as is well known in the art for such devices. By way of example only, suitable materials can include implant grade metallic materials, such as titanium, cobalt chromium alloys, stainless steel, or other suitable materials for this purpose.

Importantly, the present invention provides for the direct pressure molding of a resin composition into the tibial tray recess 20 of the tibial tray component 16 to produce a tibial tray module 12, a resin-metal composite unitary module. This innovative tibial tray module 12 results in a perfectly complementary fit of its resin and metal components and as such eliminates micro motion and the negative effects of backside wear that in conventional tibial prosthetic devices greatly reduces the effective useful life of the implant. It is also of great benefit that the keel separating notch 48a or 48b is provided so as to permit explanation of the prosthesis as needed while allowing the keel element 18 to remain firmly fixed in the calcar recess 44 of the tibia.

It is also within the concept of the present invention to provide a kit, which includes at least one modular tibial prosthesis device 10 of the present invention. Implants of different sizes can be provided in the kit to permit selection and substitution of implants or parts of implants of the correct size as needed. Additionally, a kit can include tools and/or instruments suitable to facilitate implanting the device. Such a kit can be provided with sterile packaging to facilitate opening and immediate use in an operating room.

Each of the embodiments described above are provided for illustrative purposes only and it is within the concept of the present invention to include modifications and varying configurations without departing from the scope of the invention that is limited only by the claims included herewith.

What is claimed is:

1. A novel modular tibial prosthesis comprising:
a tibial tray module, said tibial tray module comprising an articulation component formed by direct pressure molding into an underlying tibial tray component, and
an underlying keel element having an essentially flat upper surface and an under surface configured as a downward taper portion, said keel element being attached to the underside of said tibial tray component.

2. The tibial prosthesis of claim 1, wherein said tibial tray component comprises a tibial tray recess defined on an upper surface of said tibial tray component by a tray side wall, said tray side wall being circumferentially disposed and upwardly directed around the lateral limit of said tibial tray component upper surface.

3. The tibial prosthesis of claim 2, wherein said tray side wall comprises an inwardly directed retaining lip circumferentially disposed around at least a portion of said tray side wall.

4. The tibial prosthesis of claim 3, wherein said articulation component comprises a circumferentially disposed retaining groove, said retaining groove being formed by direct pressure molding of said articulation component such that said groove is sized and configure to securely seat and retain said inwardly directed retaining lip of said tibial tray component.

5. The tibial prosthesis of claim 1, wherein said tibial tray module is factory formed.

6. The tibial prosthesis of claim 5, wherein said factory formed tibial tray module is formed by direct compression molding of a resin composition for forming said articulation component into said tibial tray recess.

7. The tibial prosthesis of claim 1, wherein said underside of said tibial tray component of said tibial tray module comprises at least one trunnion and said keel element comprises an upper surface having at least one trunnion receptacle, said at least one trunnion and trunnion receptacle are of complementary number, size, configuration and respective alignment one to the other, wherein said at least one trunnion and trunnion receptacle when impacted together form a cold weld between said tibial tray component and said keel.

8. The tibial prosthesis of claim 7, wherein said at least one trunnion and said at least one trunnion receptacle are respectively three trunnions and three trunnion receptacles.

9. The tibial prosthesis of claim 7, wherein said at least one trunnion and said at least one trunnion receptacle are configured to have a Morse taper.

10. The tibial prosthesis of claim 1, wherein said underside of said tibial tray component is at least partially coated with a porous coating material.

11. The tibial prosthesis of claim 1, wherein said tibial tray component defines at least one osteotome receptacle at a position on an outer edge of said underside of said tibial tray component.

12. The tibial prosthesis of claim 1, wherein said keel element defines at least one osteotome receptacle at a position on an outer edge of said upper surface of said keel element.

13. The tibial prosthesis of claim 1, wherein said articulation component is formed of a synthetic resin composition.

14. The tibial prosthesis of claim 13, wherein said synthetic resin composition is polyethylene.

15. The tibial prosthesis of claim 1, wherein said tibial tray component is formed of materials selected from the group consisting of titanium, titanium alloys, cobalt chromium alloys, and stainless steel.

16. A method of manufacture of a tibial prosthesis, the steps of the method comprising:
providing a resin composition for forming an articulation component;
providing a tibial tray component having a tibial tray recess; and
forming said articulation component by direct pressure molding of a resin composition directly into the tibial tray recess to form a tibial tray module.

17. A kit for use in total knee arthroplasty; the kit comprising:
at one of the tibial prosthesis devices of claim 1;
at least one additional surgical tool, instrument, or implantable device.

18. The kit of claim 17, wherein two or more of the devices of claim 1 are included, said devices being of different sizes relative one to the other.

19. The kit of claim 17, further comprising sterile packaging whereby said kit can be opened directly in surgery prior to use.
